(19) United States
(12) Patent Application Publication
(10) Pub. No.: US 2015/0157610 A1
(43) Pub. Date: Jun. 11, 2015

(54) PHARMACEUTICAL COMPOSITION FOR TREATING INFLAMMATORY DISEASE

(71) Applicants: OSAKA UNIVERSITY, Osaka (JP); SHIZUOKA PREFECTURE PUBLIC UNIVERSITY CORPORATION, Shizuoka (JP)

(72) Inventors: Tetsuo Minamino, Osaka (JP); Issei Komuro, Osaka (JP); Takashi Matsuzaki, Osaka (JP); Naoto Oku, Shizuoka (JP); Tomohiro Asai, Shizuoka (JP); Haiying Fu, Osaka (JP)

(21) Appl. No.: 14/402,430

(22) PCT Filed: May 23, 2013
(86) PCT No.: PCT/JP2013/064384

§ 371 Date: Feb. 26, 2015

(30) Foreign Application Priority Data
May 23, 2012 (JP) 2012-117077

(51) Int. Cl. A61K 31/436; A61K 9/127; A61K 38/13

(52) U.S. Cl. CPC A61K 31/436; A61K 38/13; A61K 9/127

(57) ABSTRACT

A pharmaceutical composition which comprises, as an active ingredient, a liposome encapsulating an immunosuppressant such as FK506, FTY720 and cyclosporin A is effective in the treatment of cardiovascular inflammatory diseases such as my ocardial infarction, myocarditis and vasculitis syndrome, allows the immunosuppressant at a low dose to produce stronger effects than those of the same dose of the immunosuppressant used alone, and causes fewer side effects.

### TECHNICAL FIELD

The present invention relates to a pharmaceutical composition for the treatment of inflammatory diseases.

### BACKGROUND ART

Inflammatory diseases in the cardiovascular field include myocarditis, vasculitis syndrome and myocardial infarction. Myocarditis is an inflammatory disease that mainly affects the myocardium. Most types of myocarditis are caused by bacterial, viral or other infections. Known pathogens that cause myocarditis include viruses, bacteria, rickettsias, chlamydiae, spirochetes, mycoplasmas, fungi, protozoa and parasites. Besides such infections, the causes of myocarditis include drug treatment, physical stimuli such as radiation and heat, metabolic disorders, immune disorders and pregnancy. Myocarditis is classified by its histological characteristics into lymphocytic myocarditis, giant cell myocarditis, eosinophilic myocarditis and granulomatous myocarditis. Etiologically, lymphocytic myocarditis is primarily caused by viral infection, while giant cell myocarditis, eosinophilic myocarditis and granulomatous myocarditis are predominantly regarded as a complication such as cardiotoxic substances, drug allergy, autoimmunity, systemic diseases, etc. Myocardial biopsy in the early stages of the disease development makes it possible to establish a treatment program based on histological diagnosis, but in some cases, myocardial biopsy itself or accurate histological diagnosis is difficult in the early stages. In the classification by the type of onset, myocarditis is classified into acute myocarditis and chronic myocarditis. In the case of acute myocarditis, the day of symptom onset can be identified as the day of illness onset. In some cases of acute myocarditis, patients fall into a cardiopulmonary emergency in the early stages of the disease development, and this type of acute myocarditis is called fulminant myocarditis.

In patients with acute myocarditis, the onset of the disease is often preceded by cold-like symptoms (chill, fever, headache, myalgia, general malaise, etc.) and/or gastrointestinal symptoms (anorexia, nausea, vomiting, diarrhea, etc.). In subsequent several hours to several days, cardiac symptoms manifest. Fulminant myocarditis, in which cardiopulmonary emergency immediately follows simple cold symptoms and/or gastrointestinal symptoms, often has a fatal course. Currently, no effective therapy for fulminant myocarditis is available yet, and the development of novel therapies is desired. Vasculitis syndrome is caused by “inflammation” in “blood vessels”, and the patients present with symptoms relevant to ischemia and hemorrhage in multiple organs as well as symptoms of inflammation.

Giant cell myocarditis is a fatal myocarditis characterized by the appearance of a large number of multinucleated giant cells, and its clinical presentation is often similar to that of fulminant myocarditis. Non Patent Literature 1 shows the effectiveness of various immunosuppressants for the treatment of giant cell myocarditis. Further, Non Patent Literature 2 reports that the immunosuppressant FK506 (tacrolimus) is experimentally effective against fulminant myocarditis, and Non Patent Literature 3 reports that FTY720 (fingolimod) is experimentally effective against fulminant myocarditis.

Vasculitis syndrome is an inflammatory disease that mainly affects the aorta. Many types of vasculitis syndrome are rare and intractable diseases of unknown etiology and are included, as a research subject for the intractable vasculitis study group of the Ministry of Health, Labour and Welfare of Japan (MHLW), in the Specific Diseases designated by the MHLW. Among them, relatively prevalent and difficult-to-treat vasculitides are included in the disease list of the Specific Disease Treatment Research Program, under which the certified patients with such diseases are issued with medical care certificates and part of their healthcare expenses are publicly covered. Non Patent Literature 4 shows the effectiveness of various immunosuppressants for the treatment of vasculitis syndrome, and Non Patent Literature 5 reports a case in which FK506 was proven effective against Takayasu's arteritis, which is a type of vasculitis syndrome.

Myocardial infarction is a disease in which the occlusion of the coronary artery by thrombi etc. blocks the blood flow to the downstream myocardium, resulting in myocardial necrosis. A known cause of the disease is vascular inflammation resulting from infection, smoking, diabetes, hypertension, etc. The recanalization of the occluded coronary artery for blood flow restoration is known to induce free radical (e.g. reactive oxygen species) generation, vascular endothelial cell injury and inflammatory response mediated by neutrophil activation etc., resulting in additional damage to the myocardium. Non Patent Literature 6 reports that cyclosporin A reduces acute myocardial infarct size.

### CITATION LIST

### Non Patent Literature

Non Patent Literature 1:
Guidelines for Diagnosis and Treatment of Myocarditis (2009 revised edition), the Japanese Circulation Society, the Japanese Association for Thoracic Surgery, Japanese Society of Pediatric Cardiology and Cardiac Surgery, the Japanese Society for Cardiovascular Surgery, the Japanese College of Cardiology, the Japanese Heart Failure Society
Non Patent Literature 2:
Kodama M et al., Am Heart J 1993; 126(6) 1385-1392
Non Patent Literature 3:
Miyamoto T et al., J Am Coll Cardiol. 2001; 37; 1713-8
Non Patent Literature 4:
(Digest version) Guideline for Management of Vasculitis Syndrome, Circulation Journal Vol. 72, Suppl. IV, 2008, 1319-1346
Non Patent Literature 5:
Yokoe I et al., Intern Med. 2007; 46: 1873-7
Non Patent Literature 6:
Piot C et al., N Engl J Med 2008; 359; 473-481

### SUMMARY OF INVENTION

### Technical Problem

An object of the present invention is to provide a pharmaceutical composition effective in the treatment of inflammatory diseases.

### Solution to Problem

The present invention includes the following to achieve the above-mentioned object.

(1) A pharmaceutical composition for treatment of a cardiovascular inflammatory disease, comprising an immunosuppressant encapsulated liposome as an active ingredient.
(2) The pharmaceutical composition according to the above (1), wherein the cardiovascular inflammatory disease is myocarditis, vasculitis syndrome, myocardial infarction or chronic heart failure.
(3) The pharmaceutical composition according to the above (1) or (2), wherein the immunosuppressant is a steroid preparation, a calcineurin inhibitor or a sphingosine 1-phosphate receptor modulator.
(4) The pharmaceutical composition according to the above (3), wherein the immunosuppressant is FK506, FTY720 or cyclosporin A.
(5) The pharmaceutical composition according to any one of the above (1) to (4), wherein the pharmaceutical composition is used for intravenous or subcutaneous administration.
(6) The pharmaceutical composition according to the above (5), wherein the pharmaceutical composition is used for peripheral intravenous administration.
(7) The pharmaceutical composition according to the above (4), wherein the intravenous dose of a cyclosporin A encapsulated liposome per administration for a human having developed myocardial infarction is 2.0 mg/kg body weight or less.
(8) The pharmaceutical composition according to the above (4), wherein the intravenous dose of a cyclosporin A encapsulated liposome per administration for a human having developed myocarditis is 2.0 mg/kg body weight or less.
(9) The pharmaceutical composition according to the above (4), wherein the intravenous dose of an FK506 encapsulated liposome per administration for a human having developed myocarditis is 0.2 mg/kg body weight or less.
(10) A poorly water-soluble substance encapsulated liposome produced by a production method comprising the steps of:

preparing a sterol-free mixture containing a poorly water-soluble substance, phospholipids and a water miscible organic solvent so that the concentration of the poorly water-soluble substance is 0.05 mg or more relative to 1.0 mg of the phospholipids, followed by heating the mixture to give a solution,

adding, to the solution, an aqueous sugar solution, followed by mixing and heating to give a mixed solution,

heating the obtained solution, and

cooling the solution,

the liposome containing 0.05 mg or more of the poorly water-soluble substance relative to 1.0 mg of the phospholipids.
(11) A method for producing a poorly water-soluble substance encapsulated liposome containing 0.05 mg or more of a poorly water-soluble substance relative to 1.0 mg of phospholipids, the method comprising the steps of:

preparing a sterol-free mixture containing a poorly water-soluble substance, phospholipids and a water miscible organic solvent so that the concentration of the poorly water-soluble substance is 0.05 mg or more relative to 1.0 mg of the phospholipids, followed by heating the mixture to give a solution,

adding, to the solution, an aqueous sugar solution, followed by mixing and heating to give a mixed solution,

heating the obtained solution, and

cooling the solution.

### Advantageous Effects of Invention

The present invention can provide a pharmaceutical composition effective in the treatment of inflammatory diseases. The pharmaceutical composition of the present invention has the benefits of allowing a low-dose immunosuppressant to produce stronger effects than those of the same dose of the immunosuppressant used alone and causing fewer side effects. The present invention can provide a liposome containing a poorly water-soluble substance at a high concentration and a method for producing the liposome.

### BRIEF DESCRIPTION OF DRAWINGS

FIG. 1 shows the observation results of the degree of vascular permeability in the heart of a fulminant myocarditis model rat treated intravenously with fluorochrome-labeled nanoparticles.

FIG. 2 shows the measurement results of the left ventricular end-diastolic pressure in fulminant myocarditis model rats treated with an FK506 encapsulated liposome.

FIG. 3 shows the observation results of the degree of vascular permeability in the hearts of left ventricular hypertrophy-induced mice treated intravenously with fluorochrome-labeled nanoparticles. The left ventricular hypertrophy-induced mice were established by aortic arch constriction.

FIG. 4 shows the measurement results of the left ventricular end-diastolic pressure in fulminant myocarditis model rats treated with a cyclosporin A encapsulated liposome.

FIG. 5 shows the measurement results of the myocardial infarct size in acute myocardial infarction model rats treated with a cyclosporin A encapsulated liposome.

### DESCRIPTION OF EMBODIMENTS

The present invention provides a pharmaceutical composition for the treatment of inflammatory diseases, the composition comprising an immunosuppressant encapsulated liposome as an active ingredient. The immunosuppressant used for the pharmaceutical composition of the present invention is not particularly limited and known immunosuppressants can be preferably used. The known immunosuppressant include steroid preparations; antimetabolites such as azathioprine, mycophenolic acid, leflunomide, teriflunomide and methotrexate; calcineurin inhibitors such as FK506, cyclosporin A and pimecrolimus; sphingosine 1-phosphate receptor modulators such as FTY720; TNF-alpha inhibitors such as thalidomide and lenalidomide; IL-1 receptor antagonists such as anakinra; mTOR inhibitors such as rapamycin, deforolimus, everolimus, temsirolimus, zotarolimus and biolimus A9; corticosteroids such as prednisone; and various antibodies such as anti-thymocyte globulin, anti-lymphocyte globulin, anti-cytokine antibodies and anti-CD antibodies.

Preferred are steroid preparations, calcineurin inhibitors and sphingosine 1-phosphate receptor modulators, and more preferred are calcineurin inhibitors and sphingosine 1-phosphate receptor modulators. Preferable calcineurin inhibitors are FK506 and cyclosporin A, and a preferable sphingosine 1-phosphate receptor modulator is FTY720.

The liposome used for the pharmaceutical composition of the present invention is not limited as long as it is a vesicle enclosed by a lipid bilayer membrane. The liposome may be a large unilamellar vesicle (LUV), a small unilamellar vesicle (SUV) or a multilamellar vesicle (MLV). The liposome can be produced by a known production method, specifically, for example, the Bangham method, the reverse-phase evaporation method, the ultrasonic method, the extrusion method, the French press method, the homogenization method, the ethanol injection method, the dehydration-rehydration method, or the like.

The lipid used as a component of the liposome is not particularly limited, and the examples include soybean lecithin, hydrogenated soybean lecithin, yolk lecithin, phosphatidylcholines, phosphatidylserines, phosphatidylethanolamines, phosphatidylinositols, phosphasphingomyelins, phosphatidic acids, long-chain alkyl phosphates, gangliosides, glycolipids, phosphatidylglycerols and sterols. These lipids may be used alone or in a combination of two or more kinds. Examples of the phosphatidylcholines include dimyristoylphosphatidylcholine, dipalmitoylphosphatidylcholine and distearoylphosphatidylcholine. Examples of the phosphatidylserines include dipalmitoylphosphatidylserine, dipalmitoylphosphatidylserine sodium salt and bovine brain phosphatidylserine sodium salt. Examples of the phosphatidylethanolamines include dimyristoylphosphatidylethanolamine, dipalmitoylphosphatidylethanolamine and distearoylphosphatidylethanolamine. Examples of the phosphatidylinositols include wheat phosphatidylinositol sodium salt. Examples of the phosphasphingomyelins include bovine brain sphingomyelin. Examples of the phosphatidic acids and the long-chain alkyl phosphates include dimyristoyl phosphatidic acid, dipalmitoyl phosphatidic acid, distearoyl phosphatidic acid and dicetyl phosphate. Examples of the gangliosides include ganglioside GM1, ganglioside GD1a and ganglioside GT1b. Examples of the glycolipids include galactosylceramide, glucosylceramide, lactosylceramide, phosphatide and globoside. Examples of the phosphatidylglycerols include dimyristoyl phosphatidylglycerol, dipalmitoyl phosphatidylglycerol and distearoyl phosphatidylglycerol. Examples of the sterols include cholesterol, dihydrocholesterol, lanosterol, dihydrolanosterol, sitosterol, campesterol, stigmasterol, brassicasterol and ergosterol. In the case where two or more kinds of lipids are used in combination, a combination of phospholipids and cholesterol is preferable. The phospholipids are preferably phosphatidylcholines. In the case where liposomes are produced from phospholipids and cholesterol, the molar ratio of the phospholipids and the cholesterol is preferably 1:0.1 to 1.5, and more preferably 1:0.5 to 1.25.

Immunosuppressant encapsulated liposomes can be produced, according to a known method for producing liposomes, by adding an immunosuppressant solution to a solution of a lipid for forming a lipid bilayer membrane. Examples of the solvent used for a poorly water-soluble immunosuppressant such as FK506 and cyclosporin A include methanol, ethanol, isopropanol, tert-butanol and N,N-dimethylformamide. Preferred are methanol, ethanol, isopropanol and tert-butanol. Examples of the solvent used for an easily water-soluble immunosuppressant such as FTY720 include water, methanol and ethanol.

The molar ratio of the lipid and the immunosuppressant is not particularly limited, but is preferably 1:0.0001 to 0.5, more preferably 1:0.005 to 0.1, and still more preferably 1:0.001 to 0.02.

In conventional methods for producing liposomes, it has been difficult to produce liposomes containing a poorly water-soluble immunosuppressant such as FK506 and cyclosporin A at a high concentration (for example, 0.05 mg or more relative to 1.0 mg of phospholipids). A report on cyclosporin A encapsulated liposomes describes the use of liposomes containing 0.02 mg of cyclosporin A relative to 1.0 mg of phospholipids (Reference: Liposomal formulations of cyclosporin A: influence of lipid type and dose on pharmacokinetics. Fahr A, Holz M, Fricker G. Pharm Res. 1995 August; 12(8): 1189-98), but no previous reports describe the production of liposomes containing 0.05 mg or more of cyclosporin A relative to 1.0 mg of phospholipids. The present inventors have made intensive efforts to produce liposomes containing a poorly water-soluble substance at a high concentration, and as a result, succeeded in producing a liposome containing a poorly water-soluble substance at a high concentration. The increase in the content of a substance of interest relative to 1.0 mg of phospholipids can decrease the amount of liposomes used for the administration of a requisite amount of the substance. Phospholipids, ingredients of liposomes, are used as a pharmaceutical additive, but they are very expensive as a pharmaceutical additive. In addition, for administration of a larger amount of liposomes to a human than the amount used in approved drugs, extensive toxicity and safety data are required. Therefore, administration of a large amount of liposomes to a human is problematic. Using the liposome of the present invention, which contains a poorly water-soluble substance at a high concentration, is very advantageous in terms of production cost reduction and safety.

The present invention provides a poorly water-soluble substance encapsulated liposome containing 0.05 mg or more of a poorly water-soluble substance relative to 1.0 mg of phospholipids. The poorly water-soluble substance encapsulated liposome can be produced by a production method comprising the steps of:

preparing a sterol-free mixture containing a poorly water-soluble substance, phospholipids and a water miscible organic solvent so that the concentration of the poorly water-soluble substance is 0.05 mg or more relative to 1.0 mg of the phospholipids, followed by heating the mixture to give a solution,

adding, to the solution, an aqueous sugar solution, followed by mixing and heating to give a mixed solution,

heating the obtained mixture, and

cooling the mixture.

The concentration of the poorly water-soluble substance in the liposome is not particularly limited as long as it is 0.05 mg or more relative to 1.0 mg of the phospholipids. However, 0.06 mg or more relative to 1.0 mg of the phospholipids is preferred, 0.08 mg or more relative to 1.0 mg of the phospholipids is more preferred, 0.1 mg or more relative to 1.0 mg of the phospholipids is still more preferred, and 0.12 mg or more relative to 1.0 mg of the phospholipids is still more preferred.

The poorly water-soluble substance is not particularly limited and the examples include cyclosporin A, FK506, eplerenone and FTY-720. Preferred is cyclosporin A.

As the phospholipids, those exemplified in the above can be preferably used. Preferable examples of the phospholipids include hydrogenated soybean lecithin, dipalmitoylphosphatidylcholine, distearoylphosphatidylcholine, dipalmitoylphosphatidylethanolamine and dipalmitoylphosphatidylserine.

The water miscible organic solvent refers to an organic solvent that is mixable with water, such as alcohols, ethers, esters, ketones and acetals. As the water miscible organic solvent, one or more kinds of organic solvents selected from 1-propanol, isopropyl alcohol, 2-butoxyethanol and tert-butanol are preferably used.

The mixture containing a poorly water-soluble substance, phospholipids and a water miscible organic solvent is free from sterols. Particularly, the absence of cholesterol in the mixture is important. In conventional liposomes, a combination of phospholipids and cholesterol is preferably used as the lipid component, but in the liposome of the present invention, which contains a poorly water-soluble substance at a high concentration, no cholesterol is used as a lipid component. Although the reason has been poorly understood, it was found that, when no cholesterol is used as a lipid component in combination with phospholipids, the poorly water-soluble substance concentration in the mixture containing a poorly water-soluble substance, phospholipids and a water miscible organic solvent can be maintained in the liposome after encapsulation.

The sugar contained in the aqueous sugar solution is not particularly limited and, for example, monosaccharides such as glucose, and disaccharides such as maltose and sucrose can be preferably used. The sugar serves as an osmotic adjuster. The concentration of the sugar is preferably 5 to 70 wt/vol %, and more preferably 8 to 50 wt/vol % relative to the mixture after addition of the aqueous sugar solution.

The concentration of the water miscible organic solvent in the total volume of the mixture after addition of the aqueous sugar solution is preferably 5 to 30 vol %, more preferably 5 to 20 vol %, and still more preferably 12 to 20 vol %. In the case where the water miscible organic solvent is tert-butanol, its concentration in the total volume of the mixture after addition of the aqueous sugar solution is particularly preferably 12 to 18 vol %. In the case where the water miscible organic solvent is 1-propanol, its concentration in the total volume of the mixture after addition of the aqueous sugar solution is particularly preferably 5 to 19 vol %. In the case where the water miscible organic solvent is 2-propanol, its concentration in the total volume of the mixture after addition of the aqueous sugar solution is particularly preferably 13 to 26 vol %. In the case where the water miscible organic solvent is 2-butoxyethanol, its concentration in the total volume of the mixture after addition of the aqueous sugar solution is particularly preferably 6 to 9 vol %.

In the step of preparing a sterol-free mixture containing a poorly water-soluble substance, phospholipids and a water miscible organic solvent so that the concentration of the poorly water-soluble substance is 0.05 mg or more relative to 1.0 mg of the phospholipids, it is preferable that the poorly water-soluble substance, the phospholipids and the water miscible organic solvent are mixed and heated. The heating temperature is not particularly limited, but is preferably 50 to 80° C., for example. The concentration of the poorly water-soluble substance in the mixture may be set to its desired concentration in the liposome.

After the mixture containing a poorly water-soluble substance, phospholipids and a water miscible organic solvent is heat-treated for dissolution, an aqueous sugar solution is added to and mixed with the resulting solution to give a mixture containing the aqueous sugar solution. In this step, heating the obtained mixture is preferable as is the case with the previous step. The heating temperature is not particularly limited, but is preferably 50 to 80° C., for example. The thus obtained mixture is a liposome forming solution.

Optionally, but preferably, the obtained liposome forming solution is kept at a temperature lower than the heating temperature for a given period of time before cooled. The retention temperature is not particularly limited as long as it is lower than the heating temperature and allows liposome formation. However, preferred is a temperature of 40° C. or higher and lower than the heating temperature. The retention time is preferably the length of time required for the mean particle diameter of the liposomes to reach a predetermined size. In the retention step, the mixture may be cooled in two or more stages, where the respective goal temperatures are 40° C. or higher and lower than the heating temperature and the mixture is kept at each goal temperature for a given period of time.

The cooling temperature in the step of cooling the mixture (liposome forming solution) following heating is not particularly limited as long as it is a temperature lower than the heating temperature. However, preferred is a temperature of 0° C. or higher and lower than 40° C., more preferred is a temperature of 4 to 35° C., and particularly preferred is a temperature of 20 to 30° C. In the cooling step, the mixture may be left to spontaneously cool, and a cooling device may be used. The cooling step may be performed in a single stage or in two or more stages (for example, primary cooling and secondary cooling).

In the step following the preparation of the liposome forming solution, for example, a liposome assembler (Lipo-TB) manufactured by Toray Engineering Co., Ltd. is preferably used. Specifically, in a preferable example, the liposome forming solution is supplied to the liposome assembler through a tube, reheated, filter-sterilized (for example, with 0.2-μm filter) and cooled. This is a non-limiting example.

After the cooling step, it is preferable to remove the water miscible organic solvent in the obtained liposomal solution. Examples of the method for removing the water miscible organic solvent include dialysis, evaporation, drying and lyophilization.

The membrane surface of the liposome is preferably modified with a polyethylene glycol (PEG) derivative for increase of the stability of the liposome in the blood. The PEG derivative-modified liposome can be produced from a covalent complex of a phospholipid and a PEG having a molecular weight of 500 to 20000. The PEG-phospholipid covalent complex is preferably a covalent complex of distearoylphosphatidylethanolamine and a PEG having molecular weight of 2000 to 5000 (DSPE-PEG).

The size (particle diameter) of the liposome is not particularly limited, and for example, the mean particle diameter is preferably about 50 to 1000 nm, more preferably about 50 to 500 nm, still more preferably about 50 to 300 nm, and still more preferably about 75 to 200 nm. The “particle diameter” as used herein means a particle diameter measured by dynamic light scattering. The polydispersity index (PDI) is preferably 0.3 or less. The method for adjusting the particle diameter is not particularly limited. For example, a method using an extruder for several passes through a membrane filter of an appropriate pore size, a method using an ultrasonic homogenizer, and other methods can be employed.

The pharmaceutical composition of the present invention can be prepared by blending the immunosuppressant encapsulated liposome as an active ingredient, a pharmaceutically acceptable carrier and if needed an additive, and formulated into a dosage form. The dosage form is not particularly limited and may be an oral or parenteral preparation, but preferred is a parenteral preparation. Examples of the parenteral preparation include an injection, an infusion, an infusion, a suppository, an ointment, a gel, a cream, a patch, an aerosol and a spray. Among them, an injection and an infusion are preferred, and an injection and an infusion each used for intravenous administration are more preferred.

The injection may be an aqueous or oily injection. For preparation of the aqueous injection, according to a known method, for example, an immunosuppressant encapsulated liposome is mixed with a solution of an appropriate kind(s) of pharmaceutically acceptable additive(s) in an aqueous solvent (water for injection, purified water or the like), the mixture is filter sterilized with a filter or the like, and the filtrate is distributed into sterile containers. Examples of the pharmaceutically acceptable additive include isotonizing agents such as sodium chloride, potassium chloride, glycerin, mannitol, sorbitol, boric acid, borax, glucose and propylene glycol; buffering agents such as a phosphate buffer solution, an acetate buffer solution, a borate buffer solution, a carbonate buffer solution, a citrate buffer solution, a Tris buffer solution, a glutamate buffer solution and an epsilon-aminocaproate buffer solution; preservatives such as methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, disodium edetate, boric acid and borax; thickeners such as hydroxyethyl cellulose, hydroxypropyl cellulose, polyvinyl alcohol and polyethylene glycol; stabilizer such as sodium hydrogen sulfite, sodium thiosulfate, disodium edetate, sodium citrate, ascorbic acid and dibutylhydroxytoluene; and pH adjusters such as hydrochloric acid, sodium hydroxide, phosphoric acid and acetic acid. The injection may further contain an appropriate solubilizer. Examples of the solubilizer include alcohols such as ethanol; polyalcohols such as propylene glycol and polyethylene glycol; and nonionic surfactants such as polysorbate 80, polyoxyethylene hydrogenated castor oil 50, lysolecithin and Pluronic polyol. Further, proteins such as bovine serum albumin and keyhole limpet hemocyanin; polysaccharides such as aminodextran; and/or the like may be contained in the injection. For preparation of an oily injection, an oily solvent such as sesame oil and soybean oil is used, and a solubilizer such as benzyl benzoate and benzyl alcohol may be used. The prepared liquid for injection is usually distributed into appropriate ampules or vials, or other containers. Liquid preparations such as injections can be cryopreserved as they are, or preserved after deprived of water by lyophilization etc. Lyophilized preparations can be reconstituted in distilled water for injection or the like just before use.

The amount of the immunosuppressant contained in the pharmaceutical composition of the present invention varies with the dosage form and the administration route, and in the case of injections for intravenous administration for example, the amount of the immunosuppressant can be selected as appropriate within the range of 0.001 ng/mL to 100 mg/mL.

The pharmaceutical composition of the present invention can be preferably used for, the treatment of inflammatory diseases. The treatment encompasses improvement. The inflammatory diseases are not particularly limited as long as they are diseases involving inflammation. The inflammatory diseases include, for example, vascular diseases, inflammatory bowel diseases, inflammatory neurological diseases, inflammatory lung disease, inflammatory ophthalmic diseases, chronic inflammatory gingival diseases, chronic inflammatory articular disease, rheumatoid arthritis, skin diseases, bone diseases, heart diseases, renal failure, chronic demyelinating diseases, endothelial cell diseases, allergy syndrome, multiple sclerosis, skin inflammation, graft rejection, autoimmune diseases, stroke and myocardial infarction.

Preferable inflammatory diseases include vascular diseases, inflammatory bowel diseases, inflammatory neurological diseases, chronic inflammatory articular disease, rheumatoid arthritis, skin diseases, heart diseases, chronic demyelinating diseases, endothelial cell diseases, allergy syndrome, multiple sclerosis, skin inflammation, graft rejection and autoimmune diseases. More preferred are cardiovascular inflammatory diseases such as vascular diseases, inflammatory bowel diseases, inflammatory neurological diseases, heart diseases, chronic demyelinating diseases, allergy syndrome, multiple sclerosis and autoimmune diseases. The specific examples include myocarditis, vasculitis syndrome, myocardial infarction and chronic heart failure. Preferred are myocarditis, vasculitis syndrome and myocardial infarction.

The subject to be treated with the pharmaceutical composition of the present invention is preferably a mammal having developed an inflammatory disease. Examples of the mammal include a human, a monkey, a cow, a sheep, a goat, a horse, a pig, a rabbit, a dog, a cat, a rat, a mouse and a guinea pig. Particularly preferred are a human having developed an inflammatory disease and a human suspected of having developed an inflammatory disease. The administration method of the pharmaceutical composition of the present invention is not particularly limited as long as the method allows the active ingredient to reach the site of inflammation. However, preferred is parenteral administration such as intravenous administration, subcutaneous administration, intramuscular administration and intraperitoneal administration. More preferred are intravenous administration and subcutaneous administration. In the case of intravenous administration, peripheral intravenous administration is preferred. The present inventors have confirmed that peripheral intravenous administration of the pharmaceutical composition of the present invention to fulminant myocarditis model rats improves cardiac function (see Examples). That is, the pharmaceutical composition of the present invention does not require any central venous catheter for administration and thus is highly safe.

In the case where an FK506 encapsulated liposome (liposomal FK506) as the pharmaceutical composition of the present invention is intravenously administered to a human having developed myocarditis, the dose per administration is preferably 0.2 mg/kg body weight or less, more preferably 0.1 mg/kg body weight or less, still more preferably 0.05 mg/kg body weight or less, still more preferably 0.02 mg/kg body weight or less, and still more preferably 0.01 mg/kg body weight or less. Lower doses can be used without particular limitation as long as the dose can produce the desired effect. For example, the dose per administration is preferably 0.0001 mg/kg body weight or more, more preferably 0.0005 mg/kg body weight or more, and still more preferably 0.001 mg/kg body weight or more. The frequency of administration is preferably twice daily to once every three days, and more preferably once daily to once every two days.

In the case where a cyclosporin A encapsulated liposome (liposomal cyclosporin A) as the pharmaceutical composition of the present invention is intravenously administered to a human having developed myocarditis, the dose per administration is preferably 2.0 mg/kg body weight or less, more preferably 1.5 mg/kg body weight or less, still more preferably 1.0 mg/kg body weight or less, still more preferably 0.75 mg/kg body weight or less, and still more preferably 0.5 mg/kg body weight or less. Lower doses can be used without particular limitation as long as the dose can produce the desired effect. For example, the dose per administration is preferably 0.01 mg/kg body weight or more, more preferably 0.05 mg/kg body weight or more, and still more preferably 0.1 mg/kg body weight or more. The frequency of administration is preferably twice daily to once every three days, and more preferably once daily to once every two days. The intravenous administration is preferably peripheral intravenous administration.

In the case where a cyclosporin A encapsulated liposome (liposomal cyclosporin A) as the pharmaceutical composition of the present invention is intravenously administered to a human having developed myocardial infarction, the dose per administration is preferably 2.0 mg/kg body weight or less, more preferably 1.5 mg/kg body weight or less, still more preferably 1.0 mg/kg body weight or less, still more preferably 0.75 mg/kg body weight or less, and still more preferably 0.5 mg/kg body weight or less. Lower doses can be used without particular limitation as long as the dose can produce the desired effect. For example, the dose per administration is preferably 0.01 mg/kg body weight or more, more preferably 0.05 mg/kg body weight or more, and still more preferably 0.1 mg/kg body weight or more. The administration is preferably a single intravenous administration immediately before or after reperfusion treatment, and more preferably immediately before reperfusion treatment. The intravenous administration is preferably peripheral intravenous administration. In the case where free cyclosporin A is intravenously administered to a human having developed myocardial infarction, the dose per administration is usually 2.5 mg/kg body weight (see Non Patent Literature 6).

In the case where the disease to be treated is myocarditis, the type of myocarditis may be infectious myocarditis or autoimmune myocarditis, but preferred is autoimmune myocarditis. Also preferably, the type of myocarditis to be treated is giant cell myocarditis. Also preferably, the type of myocarditis to be treated is fulminant myocarditis. However, before the administration of the pharmaceutical composition of the present invention, definitive diagnosis of autoimmune myocarditis, giant cell myocarditis or fulminant myocarditis is not necessary, and a patient having developed myocarditis and a patient suspected of having developed myocarditis are preferable subjects to be treated with the pharmaceutical composition of the present invention. Here, giant cell myocarditis refers to a type of myocarditis in which many multinucleated giant cells are detected in the histological examination of myocardial biopsy specimens (see Non Patent Literature 1). Fulminant myocarditis is defined in Japan as a type of myocarditis of such a degree of severity that extracorporeal circulatory support is necessary, while the definition of fulminant myocarditis in the West includes cases where only hemodynamic support by intravenous injection of a cardiotonic drug has been provided (see Non Patent Literature 1). Many types of vasculitis syndrome are rare and intractable diseases of unknown etiology but found to involve autoimmune abnormality as a common pathological condition, and thus various immunosuppressants are shown to be effective in the treatment of vasculitis syndrome (see Non Patent Literature 4).

In the case where the disease to be treated is myocardial infarction, the pharmaceutical composition of the present invention is preferably used for the following patients with myocardial infarction.

1) Patients having developed myocardial infarction for the first time
2) Patients with ST-elevation acute myocardial infarction who received successful reperfusion treatment by transcatheter intervention within 12 hours after the onset of the disease
3) Patients having a left ventricular ejection fraction of less than 50% as determined by ultrasoundcardiography or left ventriculography before enrollment

It is recommended to avoid the use of the pharmaceutical composition of the present invention in patients obviously having reperfusion failure and in patients having Killip class III or IV or higher and cardiogenic shock at admission to a hospital.

The administration of the pharmaceutical composition of the present invention to patients with myocardial infarction can reduce reperfusion injuries in acute myocardial infarction. More specifically, for example, the effects of reducing acute myocardial infarct size, reducing lethal arrhythmias, improving myocardial stunning, preventing cardiocyte death and preventing microcirculatory obstruction can be produced.

The pharmaceutical composition of the present invention comprising an immunosuppressant encapsulated liposome as an active ingredient is useful in that an immunosuppressant at such a low dose as to be therapeutically ineffective when used in the free form achieves recovery of cardiac function inpatients with inflammatory diseases such as cardiomyopathy. That is, inflammation-induced vascular hyperpermeability in the myocarditis-affected areas and the blood vessels promotes selective accumulation of nano-size liposomes in the myocardium and the blood vessels. The liposomal encapsulation of immunosuppressants such as FK506 and FTY720 enables selective accumulation of the immunosuppressants in the lesion site, thereby potentially resulting in enhancement of drug effects and reduction of side effects. Thus, the pharmaceutical composition of the present invention is very useful. In addition, the pharmaceutical composition of the present invention can deliver an active ingredient to the target site, i.e. the site of inflammation in the myocardium by peripheral intravenous administration without the need of a central venous catheter, and thus is useful. Further, the pharmaceutical composition of the present invention is less likely to be delivered to a site other than the target site even by peripheral intravenous administration, and thus is advantageous. In summary, the pharmaceutical composition of the present invention not only allows a low-dose immunosuppressant to produce stronger effects, but also causes fewer side effects because of its reduced drug dose, its lower risk of delivery to a non-target site, the unnecessity of central venous catheters, etc., and thus is very useful.

The present invention further includes the following.

(a) A method for treating cardiovascular inflammatory diseases, comprising administering an effective amount of an immunosuppressant encapsulated liposome to a mammal.
(b) Use of an immunosuppressant encapsulated liposome for the production of a therapeutic preparation for cardiovascular inflammatory diseases.
(c) An immunosuppressant encapsulated liposome for use in the treatment of cardiovascular inflammatory diseases.

### EXAMPLES

Hereinafter, the present invention will be illustrated in detail by examples, but is not limited thereto.

### Example 1

### Preparation of FK506 Encapsulated Liposome

### (1) Preparation of Lipid Solutions and FK506 Solution

Dipalmitoylphosphatidylcholine (DPPC, Nippon Fine Chemical) was dissolved in chloroform to give a 100 mM stock solution. Distearoylphosphatidylethanolamine-methoxy PEG2000 (DSPE-mPEG2k, Nippon Fine Chemical) was dissolved in a chloroform/methanol (4/1) mixed solvent to give a 10 mM stock solution. FK506 (provided by Astellas Pharma Inc.) was dissolved in methanol to give a 1.0 mg/mL stock solution.

### (2) Preparation of FK506 Encapsulated Liposome

Preparation of an FK506 encapsulated liposome was performed so that the molar ratio of DPPC/DSPE-mPEG2K/FK506 would be 100/5/2 and that the total lipid concentration would be 10 mM. The lipid solutions and the FK506 solution were transferred with a microsyringe into an eggplant-shaped flask, and an appropriate amount of tert-butyl alcohol was added thereto. The chloroform in the mixture was removed with a rotary evaporator and the residue was frozen in liquid nitrogen. Then, overnight lyophilization was performed (EYEL-4 FDU-2200, TOKYO RIKAKIKAI CO., LTD.) for solvent removal. The lyophilized powder was hydrated with phosphate buffered saline (PBS) at 50° C. to give a liposomal FK506 solution.

In order to adjust the particle diameter of the FK506 encapsulated liposome, the liposomal FK506 solution was freeze-thawed in liquid nitrogen three times, and passed through a polycarbonate membrane filter with a pore size of 100 nm (ADVANTEC) set in an extruder (Lipex). This extrusion procedure for particle diameter adjustment was repeated 5 times or more under conditions of 50° C. to give an FK506 encapsulated liposome of about 100 nm in particle diameter,

In order to remove FK506 which had not been encapsulated in the liposome, the liposomal FK506 solution was diluted and centrifuged in an ultracentrifuge (CS120EX, HITACHI) at 453,000 g at 4° C. for 15 minutes. The supernatant was removed and the FK506 encapsulated liposome precipitated was resuspended in PBS. The thus purified FK506 encapsulated liposome was used for later experiments.

The particle diameter and the ζ potential of the FK506 encapsulated liposome were measured by Zetasizer Nano-ZS (Malvern). The amount of FK506 encapsulated in the liposome was determined by HPLC. The sample for HPLC was prepared by mixing 60 μL of the liposomal FK506 solution and 140 μL of tetrahydrofuran (THF). The HPLC measurement conditions are as follows.

### <HPLC Measurement Conditions>

### HPLC Apparatus:

Autosampler L-2200 (HITACHI)

UV detector L-2400 (HITACHI)

Pump L-2130 (HITACHI)

Column oven L-2350 (HITACHI)

### Column: TSK gel ODS-80TM (4.6×150 mm)

Mobile phase: CH₃CN/H₂O=60/40
Injection volume: 20 μL
Flow rate: 1.0 mL/min
Column temperature: 60° C.
Detection wave length: 214 nm
Measurement time: 20 min

### Example 2

### Examination of Efficacy of FK506 Encapsulated Liposome for Enhancement of Cardiac Function Improvement in Fulminant Myocarditis Model Rats

### (1) Experimental Method

### (1-1) Animals and Experimental Protocol

For induction of autoimmune myocarditis, a mixture of 0.1 mL (10 mg/mL) of porcine cardiac myosin and 0.1 mL of an adjuvant containing killed tuberculosis bacteria (10 mg/mL) was subcutaneously injected into the footpads of 7-week-old male Lewis rats to establish experimental myocarditis rats. The porcine cardiac myosin used was prepared by extraction from porcine ventricular myocardium according to a predetermined method. In order to confirm the development of myocarditis, at 21 days after the myosin injection, 0.1 mL (10 mg/mL) of fluorochrome-labeled nanoparticles (100 nm in diameter) were intravenously administered and the degree of vascular permeability in the heart was observed with a fluorescence microscope. In order to verify the therapeutic effects of FK506, at 14 and 17 days after the myosin injection, physiological saline was injected via the tail vein in a non-treatment group (control), and FK506 alone or the FK506 encapsulated liposome (0.01, 0.02, 0.05 mg/rat in terms of FK506 in both cases) was injected via the tail vein in treatment groups. At 21 days after the myosin injection, cardiac function in the rats was measured.

In another experiment, wild type mice (C57BL) were used and the aortic arch was surgically constricted for induction of left ventricular hypertrophy by pressure overload (transverse aortic constriction; TAC). At 4 weeks after TAC, fluorochrome-labeled nanoparticles were intravenously administered in the same manner as previously described and the degree of vascular permeability in the heart was observed with Ivis Lumina II imaging.

### (1-2) Cardiac Hemodynamic Measurement

At 21 days after the myosin injection, for hemodynamic parameter measurement, a catheter was inserted into the right carotid artery in the rats under anesthesia, and the left ventricular end-diastolic pressure (LVEDP) was measured as an index of heart failure.

### (1-3) Statistical Analyses

The values were given as the mean and the standard error, and statistical analyses were performed using a multi-factor analysis of variance and the Bonferroni's method. When the p value was less than 0.05, the difference was regarded as significant.

### (2) Experimental Results

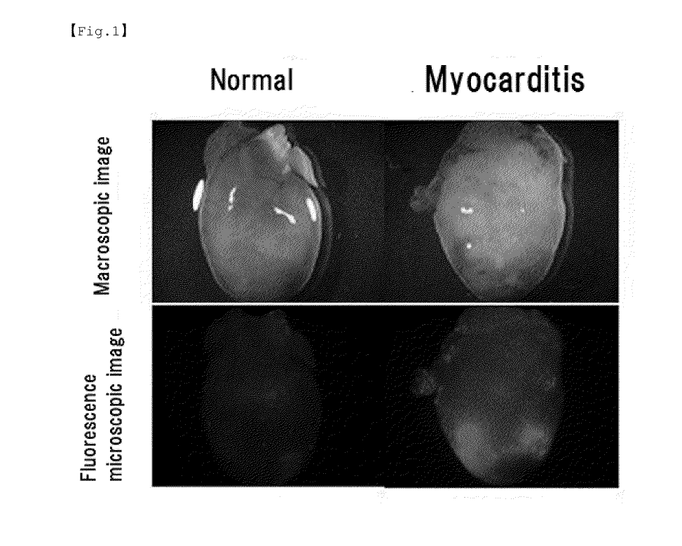

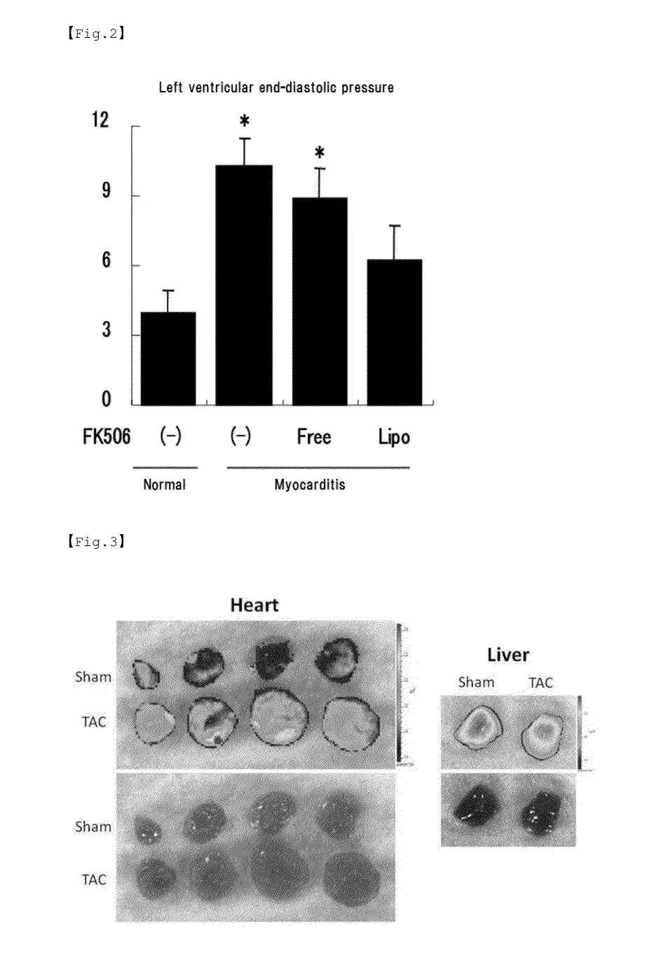

The results are shown in FIGS. 1, 2 and 3. As is clear from FIG. 1, the fluorescence intensity in the heart of the myosin injected rat was remarkably stronger than that in the normal heart, demonstrating nanoparticle accumulation and vascular hyperpermeability in the myocarditis heart. As is clear from FIG. 2, the left ventricular end-diastolic pressure as a hemodynamic measure of cardiac function was significantly increased in the myosin injected rats at 21 days post-injection as compared with the normal rats. The administration of free-FK506 (0.01 mg/rat) did not result in improvement in the left ventricular end-diastolic pressure. On the other hand, the low-dose administration of lipo-FK506 (0.01 mg/rat) significantly improved the left ventricular end-diastolic pressure. In the medium-dose (0.02 mg/rat) and high-dose (0.05 mg/rat) administrations of free-FK506, the left ventricular end-diastolic pressure was significantly improved (data not shown). However, regarding the degree of the improvement, the medium-dose administration of free-FK506 was inferior to the low-dose administration of lipo-FK506, and even the high-dose administration of free-FK506 was comparable to the low-dose administration of lipo-FK506. On the other hand, the results of the medium-dose and high-dose administrations of lipo-FK506 showed that the improvement in the left ventricular end-diastolic pressure was enhanced in a dose dependent manner. As is clear from FIG. 3, vascular hyperpermeability was observed in the failing hearts.

### Example 3

### Preparation of Cyclosporin a Encapsulated Liposome

### (1) Preparation of Lipid Solutions and Cyclosporin Solution

In 10 mL of isopropanol, 383.2 mg of hydrogenated soybean phospholipid (hydrogenated soybean phosphatidylcholine; HSPC, Nippon Fine Chemical), 127.6 mg of distearoylphosphatidylethanolamine-methoxy PEG2000 (DSPE-mPEG2k, Nippon Fine Chemical) and 40.0 mg of cyclosporin A were suspended, and then dissolved under heating at 80° C. to give a cyclosporin A/lipid solution (1 mg/mL).

### (2) Preparation of Cyclosporin A Encapsulated Liposome

Preparation of a cyclosporin A encapsulated liposome was performed so that the molar ratio of HSPC/DSPE-mPEG2K/cyclosporin A would be 14.7/1.4/1 and that the total lipid concentration would be 13.3 mM. The cyclosporin A/lipid solution was mixed with a 20 mL of a maltose-containing mixed solution (mixed solution of 250 mL of 10% maltose, 5.0 mL of 0.5 M sodium phosphate (pH 6.5) and 7.0 mL of 50% glucose), and the mixture was heated at 80° C. for dissolution to give a liposome forming solution. The liposome forming solution was supplied by a peristaltic pump (KrosFlo KR2i, Spectrum Laboratories, Inc.) to a liposome assembler (Lipo-TB, Toray Engineering) and subjected to a series of reaction steps of heating (80° C.), filter sterilization, primary cooling (20° C.) and secondary cooling (20° C.) in the flow passage in the apparatus to give a liposomal solution. The liposomal solution was supplied to a hollow fiber membrane module (mPES 500 kDa, Spectrum Laboratories, Inc.) and subjected to counter-current dialysis using, as a dialysate, a mixed solution of 250 mL of 10% maltose and 5.0 mL of 0.5 M sodium phosphate (pH 6.5) for high-speed removal of isopropanol from the liposomal solution.

The particle diameter and the potential of the cyclosporin A encapsulated liposome were measured by Zetasizer Nano-ZS (Malvern). The phospholipid concentration was measured with a commercial diagnostic kit “phospholipid C-Test Wako” (Wako Pure Chemical Industries, Ltd.) according to the manufacturer's protocol. The amount of cyclosporin A encapsulated in the liposome was determined by HPLC. The sample for HPLC was prepared by mixing 10 μL of the liposomal cyclosporin A solution and 500 μL of methanol. The HPLC measurement conditions are as follows.

### <HPLC Measurement Conditions>

### HPLC Apparatus:

Autosampler 3023 (Shiseido)

UV detector 3117 (Shiseido)

Pump 3301 (Shiseido)

Column oven 3004 (Shiseido)

### Column: Vydac C18 (4.6×250 mm)

Mobile phase: 0.01 M TFA/CH₃CN 30:70
Injection volume: 20 μL
Flow rate: 1.0 mL/min
Column temperature: 60° C.
Detection wave length: 215 nm
Measurement time: 20 min

The measurement results are as follows.

Cyclosporin A: 1.37 mg/mL
Phospholipids: 9.81 mg/mL
The amount of cyclosporin A relative to 1 mg of the
phospholipids: 0.14 mg
Particle diameter (Z-Average): 87 nm
ζ potential: −55 mV

### Example 4

### Examination of Efficacy of Cyclosporin a Encapsulated Liposome for Enhancement of Cardiac Function Improvement in Fulminant Myocarditis Model Rats

The same experiment as in Example 2 was performed using the cyclosporin A encapsulated liposome prepared in Example 3 instead of the FK506 encapsulated liposome. Three administration groups shown below were prepared.

Physiological saline administration group

Cyclosporin A (0.1 mg/kg) administration group

Cyclosporin A encapsulated liposome (0.1 mg/kg) administration group

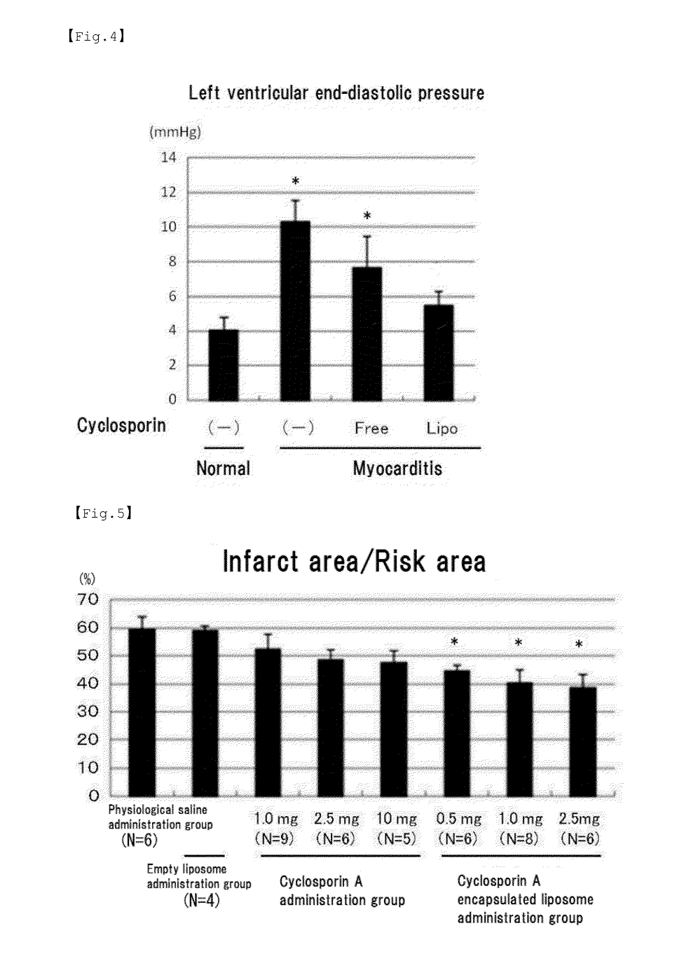

The results are shown in FIG. 4. As is clear from FIG. 4, the left ventricular end-diastolic pressure as a hemodynamic measure of cardiac function was significantly increased in the myosin injected rats at 21 days post-injection as compared with the normal rats. The cyclosporin A (0.1 mg/kg) administration group showed no improvement in the left ventricular end-diastolic pressure. On the other hand, the cyclosporin A encapsulated liposome (0.1 mg/kg) administration group showed significant improvement in the left ventricular end-diastolic pressure.

### Example 5

### Examination of Efficacy of Cyclosporin a Encapsulated Liposome for Myocardial Infarct Size Reduction in Acute Myocardial Infarction Model Rats

### (1) Experimental Method

### (1-1) Animals and Experimental Protocol

Eight to nine-week-old male rats were intraperitoneally anesthetized with an anesthetic mixture of medetomidine (Domitor, Nippon Zenyaku Kogyo Co., Ltd., 0.15 mg/kg), midazolam (Dormicum, Astellas Pharma Inc., 2 mg/kg) and butorphanol (Vetorphale, Meiji Seika Pharma Co., Ltd., 2.5 mg/kg), fixed in the supine position, orotracheally intubated for mechanical ventilation using a ventilator for small animals (Model SIN-480-7, Shinano Manufacturing Co., Ltd.) (tidal volume: 1.5 to 2.0 mL/stroke, respiration rate: 80 strokes/min), and subjected to lateral thoracotomy for exposure of the heart. The left anterior descending artery (LAD) of each rat was occluded with a needled suture (ELP, ELP needled suture: M10-50B2) for 30 minutes. During the time, electrocardiogram (lead II) was measured by TRANSDUCER Control unit (Millar, Model TCB-500), and based on the change in ST-segment potential and the color of the myocardium, the presence of occlusion was confirmed. After the 30-minute occlusion, reperfusion was started and maintained for 90 minutes for restoration of the blood flow to establish myocardial ischemia-reperfusion model rats.

Eight administration groups shown below were prepared.

Physiological saline administration group

Empty liposome administration group

Cyclosporin A (1.0 mg/kg) administration group

Cyclosporin A (2.5 mg/kg) administration group

Cyclosporin A (10.0 mg/kg) administration group

Cyclosporin A encapsulated liposome (0.5 mg/kg in terms of cyclosporin A) administration group

Cyclosporin A encapsulated liposome (1.0 mg/kg in terms of cyclosporin A) administration group

Cyclosporin A encapsulated liposome (2.5 mg/kg in terms of cyclosporin A) administration group

Physiological saline, a cyclosporin A solution or a cyclosporin A encapsulated liposome was continuously administered with a syringe pump (NIHON KOHDEN syringe pump, CV-3200) through a catheter (INTRAMEDIC Polyethylene Tubing PE50, CLAYADAMS) previously inserted into the femoral vein.

### (1-2) Hemodynamic Measurement

The blood pressure (systolic blood pressure (SBP)) and the heart rate were measured with Power Lab (AD Instruments, Castle Hill, Australia) through a catheter (Millar, MIKRO-TIP CATHETER TRANSDUCERS, Model SPR-320, size 2F) previously inserted in the carotid artery. The measurement was performed before the infarction, during the infarction and after the reperfusion, and the data were recorded. The heart rate was measured immediately before the infarction, immediately before the reperfusion, and 30, 60 and 90 minutes after the reperfusion, and determined as an average of 10 consecutive beats at each measurement time point.

### (1-3) Myocardial Infarct Size Measurement

The LAD in the heart of each rat was re-ligated at the previously occluded site after the 90-minute reperfusion, and 1 mL of a 5% Evans Blue solution (solution in physiological saline, Nacalai Tesque, Inc.) was injected into the femoral vein to stain the non-ischemic area. Each rat was euthanized with an overdose of the same anesthetic mixture as previously mentioned, and the heart was resected and immediately immersed in physiological saline (solution temperature: 37° C.) for rinsing. The heart was cut into four transverse slices of the same thickness from immediately below the LAD occlusion site to the apex. For identification of the myocardial infarct area (MI area), the slices were stained with a 1% TTC (2,3,5-triphenyltetrazolium hydrochloride, Sigma Chemical Co.) solution (solution in a phosphate buffer (pH 7.4) (Wako Pure Chemical Industries)) at a solution temperature of 37° C. for 5 minutes. The right ventricle was separated from each slice after the staining and the specimen was photographed under a stereomicroscope (OLYMPUS SZX12). The images of the photographs were captured by an image analyzer (equipped with general-purpose image-processing software Image J 1.42q), and the dimension measurement was performed on four slices per animal (one cut surface of the slice nearest to the apex and both (top and bottom) cut surfaces of each of the other three slices, and that is seven cut surfaces in total) to calculate the percentage of the ischemic area in the left ventricle (risk area/LV area (Risk/LV): %) and the myocardial infarct size (MI area/risk area (MI/Risk) and MI area/LV area (MI/LV): %). For the calculation, the LV area, the risk area and the MI area of each cut surface were each measured, and the total area for each parameter was calculated by summation across all the cut surfaces. The percentage of the ischemic area (risk area/LV area (Risk/LV): %) was determined as total risk area/total LV area, and the myocardial infarct (MI) size (MI area/risk area (MI/Risk) and MI area/LV area (MI/LV): %) was determined as total MI area/total risk area.

### (1-4) Statistical Analysis Method

The experimental data (blood pressure, heart rate and myocardial infarct size) were shown as the mean±standard error (S.E.). Statistical analyses were performed based on a two-way analysis of variance and the Bonferroni's method.

### (2) Experimental Results

The results are shown in FIG. 5. As is clear from FIG. 5, all the three cyclosporin A encapsulated liposome administration groups (0.5, 1.0 and 2.5 mg/kg) showed significant reduction in myocardial infarct size. No significant difference was observed among the three groups. On the other hand, the cyclosporin A administration groups showed a downward tendency in myocardial infarct size as compared with the physiological saline group, but no significant difference from the control was observed.

The present invention is not limited to particular embodiments and examples described above, and various modifications can be made within the scope of the appended claims. Other embodiments provided by suitably combining technical means disclosed in separate embodiments of the present invention are also within the technical scope of the present invention. All the academic publications and patent literature cited in the description are incorporated herein by reference.

1-9. (canceled)

10. A poorly water-soluble substance encapsulated liposome produced by a production method comprising the steps of:
preparing a sterol-free mixture containing a poorly water-soluble substance, a phospholipid and a water miscible organic solvent so that the concentration of the poorly water-soluble substance is 0.05 mg or more relative to 1.0 mg of the phospholipid, followed by heating the mixture to give a solution,
adding, to the solution, an aqueous sugar solution, followed by mixing and heating to give a mixed solution,
heating the obtained solution, and
cooling the solution,
the liposome containing 0.05 mg or more of the poorly water-soluble substance relative to 1.0 mg of the phospholipid.

11. A method for producing a poorly water-soluble substance encapsulated liposome containing 0.05 mg or more of a poorly water-soluble substance relative to 1.0 mg of phospholipid, the method comprising the steps of:
preparing a sterol-free mixture containing a poorly water-soluble substance, a phospholipid and a water miscible organic solvent so that the concentration of the poorly water-soluble substance is 0.05 mg or more relative to 1.0 mg of the phospholipid, followed by heating the mixture to give a solution,
adding, to the solution, an aqueous sugar solution, followed by mixing and heating to give a mixed solution,
heating the obtained solution, and
cooling the solution.

12. A method for treating a cardiovascular inflammatory disease selected from myocarditis, myocardial infarction and chronic heart failure, the method comprising administering an effective amount of an immunosuppressant encapsulated liposome to a mammal.

13. The method according to claim 12, wherein the cardiovascular inflammatory disease is chronic heart failure.

14. The method according to claim 12, wherein the immunosuppressant is a steroid preparation, a calcineurin inhibitor or a sphingosine 1-phosphate receptor modulator.

15. The method according to claim 12, wherein the immunosuppressant is FK506, FTY720 or cyclosporin A.

16. The method according to claim 12, wherein the administration is intravenous or subcutaneous administration.

17. The method according to claim 16, wherein the administration is peripheral intravenous administration.

18. The method according to claim 12, wherein the intravenous dose of a cyclosporin A encapsulated liposome per administration for a human having developed myocardial infarction is 2.0 mg/kg body weight or less in terms of cyclosporin A.

19. The method according to claim 12, wherein the intravenous dose of a cyclosporin A encapsulated liposome per administration for a human having developed myocardial infarction is 1.0 mg/kg body weight or less in terms of cyclosporin A.

20. The method according to claim 12, wherein the intravenous dose of a cyclosporin A encapsulated liposome per administration for a human having developed myocardial infarction is 0.5 mg/kg body weight or less in terms of cyclosporin A.

21. The method according to claim 12, wherein the intravenous dose of a cyclosporin A encapsulated liposome per administration for a human having developed myocarditis is 0.5 mg/kg body weight or less in terms of cyclosporin A.

22. The method according to claim 12, wherein the intravenous dose of a cyclosporin A encapsulated liposome per administration for a human having developed myocarditis is 0.1 mg/kg body weight or less in terms of cyclosporin A.

23. The method according to claim 12, wherein the intravenous dose of an FK506 encapsulated liposome per administration for a human having developed myocarditis is 0.05 mg/kg body weight or less in terms of FK506.

24. The method according to claim 12, wherein the intravenous dose of an FK506 encapsulated liposome per administration for a human having developed myocarditis is 0.02 mg/kg body weight or less in terms of FK506.

25. The liposome according to claim 10, wherein the poorly water-soluble substance is an immunosuppressant selected from FK506, FTY720 and cyclosporin A.

26. A pharmaceutical composition for treatment of a cardiovascular inflammatory disease selected from myocarditis, myocardial infarction and chronic heart failure, the pharmaceutical composition comprising the liposome according to claim 25.

27. A method for treating myocarditis, myocardial infarction or chronic heart failure, comprising administering an effective amount of the liposome according to claim 25 to a mammal.

28. The method according to claim 27, wherein the administration is intravenous or subcutaneous administration.

29. The method according to claim 28, wherein the administration is peripheral intravenous administration.

30. The method according to claim 27, wherein the intravenous dose of a cyclosporin A encapsulated liposome per administration for a human having developed myocardial infarction is 2.0 mg/kg body weight or less in terms of cyclosporin A.

31. The method according to claim 27, wherein the intravenous dose of a cyclosporin A encapsulated liposome per administration for a human having developed myocardial infarction is 1.0 mg/kg body weight or less in terms of cyclosporin A.

32. The method according to claim 27, wherein the intravenous dose of a cyclosporin A encapsulated liposome per administration for a human having developed myocardial infarction is 0.5 mg/kg body weight or less in terms of cyclosporin A.

33. The method according to claim 27, wherein the intravenous dose of a cyclosporin A encapsulated liposome per administration for a human having developed myocarditis is 0.5 mg/kg body weight or less in terms of cyclosporin A.

34. The method according to claim 27, wherein the intravenous dose of a cyclosporin A encapsulated liposome per administration for a human having developed myocarditis is 0.1 mg/kg body weight or less in terms of cyclosporin A.

35. The method according to claim 27, wherein the intravenous dose of an FK506 encapsulated liposome per administration for a human having developed myocarditis is 0.05 mg/kg body weight or less in terms of FK506.

36. The method according to claim 27, wherein the intravenous dose of an FK506 encapsulated liposome per administration for a human having developed myocarditis is 0.02 mg/kg body weight or less in terms of FK506.
